(19) United States
(12) Patent Application Publication
(10) Pub. No.: US 2013/0284165 A1
(43) Pub. Date: Oct. 31, 2013

(54) SYSTEM AND METHOD FOR TREATING A MEDICAL CONDITION USING AN AEROSOLIZED SOLUTION

(71) Applicant: William S. Krimsky, Owing Mills, OH (US)

(72) Inventor: William S. Krimsky, Owing Mills, OH (US)

(73) Assignee: MEDSTAR HEALTH, Columbia, MD (US)

(21) Appl. No.: 13/871,291

(22) Filed: Apr. 26, 2013

Related U.S. Application Data
(60) Provisional application No. 61/639,338, filed on Apr. 27, 2012.

(51) Int. Cl. A61M 15/00

(52) U.S. Cl. CPC A61M 15/0065
USPC 128/200.14

(57) ABSTRACT

A system for treating a medical condition in a subject using an aerosolized solution includes a subject interface, an inspiratory limb in fluid communication with the subject interface, an expiratory limb in fluid communication with the subject interface, and a controller in electrical communication with the inspiratory and expiratory limbs. The inspiratory limb includes an aerosol-generating device configured to provide the aerosolized solution. The controller is configured to automatically regulate at least one treatment parameter based on feedback from one or more sensors operably integrated into the inspiratory limb and/or the expiratory limb. The at least one treatment parameter is selected from the group consisting of the amount, rate, and temperature of the aerosolized solution.

### RELATED APPLICATION

This application claims the benefit of U.S. Provisional Patent Application Ser. No. 61/639,338, filed Apr. 27, 2012, the entirety of which is hereby incorporated by reference for all purposes.

### TECHNICAL FIELD

The present disclosure relates generally to a system and method for precise and controlled delivery of an aerosolized solution to a subject, and more particularly to a closed-loop system and method for selectively modulating the body temperature of a subject using an aerosolized solution.

### BACKGROUND

Hypothermia is routinely induced by physicians to protect the heart and brain of patients during cardiac surgery or operations involving cerebral blood vessels. Physicians may also rapidly cool a patient's body to protect brain tissue following traumatic injury, during resuscitation from cardiac arrest, and to help prevent brain damage after a stroke. In other instances, the rapid warming of a patient can be important, e.g., in cases where hypothermia has resulted from an accident.

At present, cardio-pulmonary bypass (CPB) is the most effective method for rapidly changing a patient's core temperature. However, CPB is invasive and requires sophisticated equipment and well-trained personnel. Non-invasive approaches to changing core temperature currently in use rely upon surface cooling or heating by covering a patient's body with a blanket in which either air or water is circulated.

Another approach has been to use the respiratory system for heat exchange. Because liquids generally have higher specific heats than gases, ventilation of patients with a liquid provides one attractive alternative for controlling body temperature. However, minute ventilation with a liquid is limited by its high viscosity and this, in turn, leads to severe CO₂ retention by patients. Moreover, liquids tend to wash out surfactants from the alveoli of lungs, thereby causing injury. A gas can be used for inhalation, but delivery of gases tends to be low and, consequently, heat exchange is relatively slow. All of these treatment modalities, however, require significant endovascular devices or cumbersome external components (e.g., mats), which decreases their ease of use and efficiency.

### SUMMARY

According to one aspect of the present disclosure, a system is provided for treating a medical condition in a subject using an aerosolized solution. The system includes a subject interface, an inspiratory limb in fluid communication with the subject interface, an expiratory limb in fluid communication with the subject interface, and a controller in electrical communication with the inspiratory and expiratory limbs. The inspiratory limb includes an aerosol-generating device configured to provide the aerosolized solution. The controller is configured to automatically regulate at least one treatment parameter based on feedback from one or more sensors operably integrated into the inspiratory limb and/or the expiratory limb. The at least one treatment parameter is selected from the group consisting of the amount, rate, and temperature of the aerosolized solution.

According to another aspect of the present disclosure, a method is provided for treating a medical condition in a subject. One step of the method comprises providing a system including a subject interface, an inspiratory limb in fluid communication with the subject interface, an expiratory limb in fluid communication with the subject interface, and a controller in electrical communication with the inspiratory and expiratory limbs. The subject interface is operably coupled to the subject. Next, an aerosolized solution is delivered from the system to the subject in an amount and for a time sufficient to treat the medical condition. The controller automatically regulates at least one treatment parameter based on feedback from one or more sensors operably integrated into the inspiratory limb and/or the expiratory limb. The at least one treatment parameter is selected from the group consisting of the amount, rate, and temperature of the aerosolized solution.

According to another aspect of the present disclosure, a system is provided for regulating the body temperature of a subject using an aerosolized solution. The system can comprise a subject interface, an inspiratory limb, an expiratory limb, a controller, and a Y-connector. The inspiratory limb can be in fluid communication with the subject interface. The inspiratory limb includes an aerosol-generating device configured to provide the aerosolized solution. The expiratory limb can be in fluid communication with the subject interface. The controller can be in electrical communication with the inspiratory and expiratory limbs. The controller can be configured to automatically regulate at least one treatment parameter based on feedback from one or more sensors operably integrated into the inspiratory limb and/or the expiratory limb. The at least one treatment parameter can be selected from the group consisting of the amount, rate, and temperature of the aerosolized solution. The Y-connector can have a first port configured to mate with the subject interface, a second port configured to mate with an end of the expiratory limb, and a third port configured to mate with an end of the aerosol-generating device. The aerosol-generating device can further include an aerosol-generating mechanism to improve aerosolization of a therapeutic liquid or solution and increase delivery efficiency of the aerosolized solution into the subject.

### BRIEF DESCRIPTION OF THE DRAWINGS

The foregoing and other features of the present disclosure will become apparent to those skilled in the art to which the present disclosure relates upon reading the following description with reference to the accompanying drawings, in which:

FIG. 1 is a schematic illustration showing a system for treating a medical condition in a subject using an aerosolized solution constructed in accordance with one aspect of the present disclosure;

FIG. 2A is a schematic illustration showing an exploded view of a Y-connector, a subject interface, an inspiratory limb, an expiratory limb, and an aerosol-generating device constructed in accordance with another aspect of the present disclosure;

FIG. 2B is a schematic illustration showing an assembled view of the Y-connector, the subject interface, the inspiratory limb, the expiratory limb, and the aerosol-generating device in FIG. 2A;

FIG. 3A is a schematic illustration showing an exploded view of a Y-connector, a subject interface, an inspiratory limb, an expiratory limb, an adaptor, and an aerosol-generating device constructed in accordance with another aspect of the present disclosure;

FIG. 3B is a schematic illustration showing an assembled view of the Y-connector, the subject interface, the inspiratory limb, the expiratory limb, the adaptor, and the aerosol-generating device in FIG. 3A;

FIG. 4A is a schematic illustration showing an exploded view of a Y-connector, a subject interface, an inspiratory limb, an expiratory limb, an H-shaped adaptor, and an aerosol-generating device constructed in accordance with another aspect of the present disclosure;

FIG. 4B is a schematic illustration showing an assembled view of the Y-connector, the subject interface, the inspiratory limb, the expiratory limb, the H-shaped adaptor, and the aerosol-generating device in FIG. 4A;

FIG. 5 is a schematic illustration showing an alternative configuration of the system in FIG. 1;

FIG. 6A is a magnified view of the system in FIG. 5 showing operation of a valve during inspiration;

FIG. 6B shows operation of the valve in FIG. 6A during expiration;

FIG. 7 is a process flow diagram illustrating a method for treating a medical condition in a subject according to another aspect of the present disclosure; and

FIG. 8 is a process flow diagram illustrating another method for treating a medical condition in a subject according to an aspect of the present disclosure.

### DETAILED DESCRIPTION

Unless otherwise defined, all technical terms used herein have the same meaning as commonly understood by one of ordinary skill in the art to which the present disclosure pertains.

In the context of the present disclosure, the term “medical condition” can refer to disease or conditions the treatment of which may be facilitated or improved by delivery of an aerosolized solution to the airway and/or lung(s) of a subject. In some instances, a medical condition can include a condition or disease that requires modulating (e.g., increasing or decreasing) body temperature, such as inducing hypothermia to treat ischemic events (e.g., heart attack, stroke, etc.), trauma (e.g., traumatic brain injury), or during prolonged surgery. In other instances, a medical condition can include an acute or chronic disease, such as cancer or asthma. In further instances, a medical condition can include a central nervous system bleed or shock.

As used herein, the term “subject” can refer to any warm-blooded organism including, but not limited to, human beings, pigs, rats, mice, dogs, goats, sheep, horses, monkeys, apes, rabbits, cattle, etc.

As used herein, the term “aerosol” can refer to a liquid or particulate matter dispersed in air. Aerosols can include dispersions of liquids (including aqueous and other solutions) and solids (including powders) in air. In some instances, an aerosol can be generated by an aerosol-generating device, such as a nebulizer or an atomizer.

As used herein, the term “nebulizer” can refer to any instrument capable of generating very fine liquid droplets for inhalation into the airway and/or lung(s). Within a nebulizer, a liquid or solution can be atomized into a mist of droplets with a broad size distribution by, for example, compressed air, ultrasonic waves, or a vibrating orifice. Nebulizers may further contain, e.g., a baffle which, along with the housing of the instrument, selectively removes large droplets from the mist by impaction. Thus, the mist inhaled into the airway and/or lung(s) can contain fine aerosol droplets. A further description of nebulizers contemplated by the present disclosure is provided below.

As used herein, the term “atomizer” can refer to any device capable of converting a solution or liquid into a fine mist spray. Unlike a nebulizer, an atomizer can deliver liquid-to-mist instantly instead of over a period of time (e.g., about 10 minutes). Also unlike a nebulizer, an atomizer is capable of dispensing the mist in small, controlled and metered amounts.

As used herein, the term “aerosolized solution” can refer to a solution that is dispersed in air to form an aerosol. Thus, an aerosolized solution can include a particular form of an aerosol.

As used herein, the terms “treat” or “treatment” can refer to any means and manner in which one or more of the symptoms of a medical condition, disorder or disease is ameliorated or otherwise beneficially altered. Amelioration of the symptoms of a particular medical condition by treatment with the present disclosure can refer to any lessening, whether permanent or temporary, lasting or transient that can be attributed to or associated with the present disclosure.

As used herein, the terms “connected”, “coupled”, and “communication” can refer to any form of interaction between two or more entities or components, including mechanical, electrical, magnetic, electromagnetic, fluid and thermal interaction. Two components may be coupled to each other even though they are not in direct contact with each other. For example, two components may be coupled to each other through an intermediate component. In some instances, the terms can refer to a form of interaction between two or more entities or components whereby the entities or components are directly coupled to each other without any intervening or intermediate component(s) therebetween. For example, it will be understood that when an element is referred to as being “on,” “attached” to, “connected” to, “coupled” with, “contacting,” etc., another element, it can be directly on, attached to, connected to, coupled with or contacting the other element or intervening elements may also be present. In contrast, when an element is referred to as being, for example, “directly on,” “directly attached” to, “directly connected” to, “directly coupled” with or “directly contacting” another element, there are no intervening elements present. It will also be appreciated by those of skill in the art that references to a structure or feature that is disposed “directly adjacent” another feature may have portions that overlap or underlie the adjacent feature, whereas a structure or feature that is disposed “adjacent” another feature may not have portions that overlap or underlie the adjacent feature.

As used herein, the term “electrical communication” can refer to the ability of a generated electric field to be transferred to, or have an effect on, one or more components of the present invention. In some instances, the generated electric field can be directly transferred to a component (e.g., via a wire or lead). In other instances, the generated electric field can be wirelessly transferred to a component.

As used herein, the term “fluid communication” can refer to two chambers, vessels, lines, tubes, pipes, tanks, or other structures containing a fluid, such as a liquid or gas, where the fluid-containing structures are connected together (e.g., by a line, pipe or tubing) so that a fluid can flow between the two fluid-containing structures. Therefore, two structures that are in “fluid communication” can, for example, be connected together by a line between the two structures, such that a fluid can flow freely between the two structures. Alternatively, two structures can be directly connected to one another so that a fluid can flow freely between the two structures.

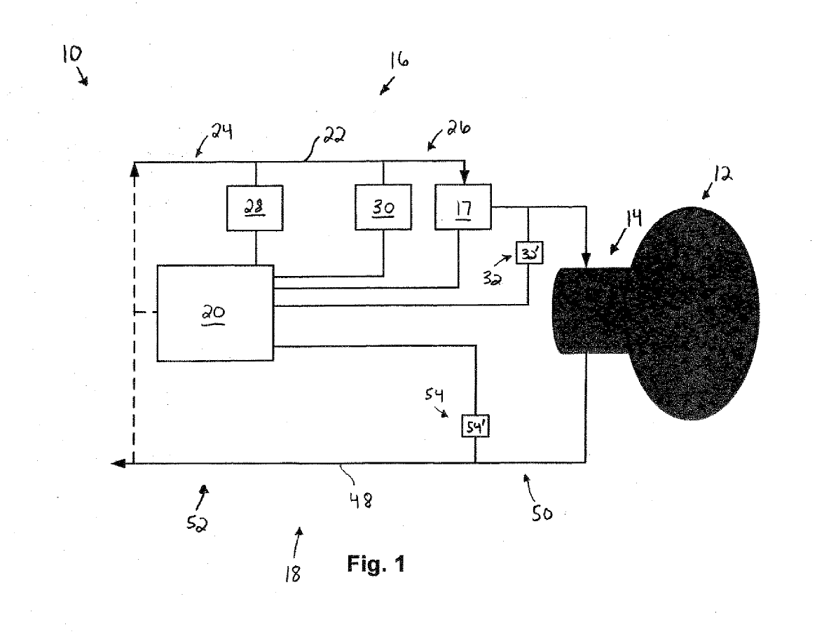

The present disclosure relates generally to a system and method for precise and controlled delivery of an aerosolized solution to a subject, and more particularly to a closed-loop system and method for selectively modulating the body temperature of a subject using an aerosolized solution. As representative of one aspect of the present disclosure, FIG. 1 illustrates a system 10 for treating a medical condition in a subject 12 using an aerosolized solution. Conventional metabolic cooling apparatus require invasive endovascular devices or cumbersome components, which decrease their ease of use and efficiency. Advantageously, the present disclosure provides a simple closed-loop system 10 configured to precisely and efficiently control delivery of an aerosolized solution to a subject 12. Although the present disclosure is described primarily in terms of modulating body temperature (e.g., inducing hypothermia), it will be appreciated that the present disclosure may be used to treat a variety of other medical conditions, such as those listed above.

As shown in FIG. 1, one aspect of the present disclosure includes a system 10 for treating a medical condition in a subject 12 using an aerosolized solution. The system 10 generally includes a subject interface 14, an inspiratory limb 16 in fluid communication with the subject interface, an aerosol-generating device 17 operably coupled or connected to the inspiratory limb, an expiratory limb 18 in fluid communication with the subject interface, and a controller 20 in electrical communication with the inspiratory limb and the expiratory limb. Although reference to FIG. 1 will be made when describing the system 10 and its components, it will be appreciated that the number and arrangement of the system components can be differently configured than is shown in FIG. 1, so long as the system is capable of providing effective treatment of a medical condition using an aerosolized solution.

In another aspect, the inspiratory limb 16 of the system 10 can include a first conduit 22 having oppositely disposed first and second end portions 24 and 26. In some instances, the first conduit 22 can comprise a flexible or flexibly resilient tube or line (e.g., plastic medical tubing) of a suitable length and size (e.g., diameter). The first end portion 24 of the first conduit 22 can be fluidly connected or coupled to at least one source 28 of a physiologically-acceptable therapeutic gas and/or liquid. In some instances, the source 28 can include a pressurized tank (not shown) containing a coolant or cryogen. In one example, the source 28 can include a pressurized tank containing a therapeutic gas, such as oxygen, nitric oxide, nitrogen dioxide, nitrogen or air. In another example, the source 28 can include a pressurized tank of a therapeutic liquid, such as liquid oxygen, liquid nitrogen, perfluorocarbons, saline or lactate ringers.

In another aspect, a heating and/or cooling device 30 can be operably connected or coupled to the first conduit 22. In one example, the heating and/or cooling device 30 can be located downstream of the source 28. The heating and/or cooling device 30 can include any known device or apparatus capable of heating or cooling the therapeutic gas and/or liquid. For example, a heater (not shown) can be operably coupled or connected to the first conduit 22 downstream of the source 28. In another example, a cooler (not shown) can be operably coupled or connected to the first conduit 22 downstream of the source 28.

In another aspect, the inspiratory limb 16 can include one or more integrated sensors 32. In some instances, the sensor(s) 32 can be physically integrated into the first conduit 22. The sensor(s) 32 is/are in electrical communication with the controller 20. The sensor(s) 32 can be configured to provide a feedback signal (or signals) to the controller 20. The feedback signal(s) can be indicative of at least one treatment parameter, such as the amount, rate and/or temperature of the aerosolized solution. In one example, a first sensor 32′ can comprise a thermistor that is located downstream of the aerosol-generating device 17 and in electrical communication with the controller 20. Other parameters that may be detected can include gas concentration(s), inspiratory limb pressure, vapor content, etc. It will be appreciated that other sensors 32 can be incorporated into the inspiratory limb 16 at different locations, such as immediately downstream from the source 28 (e.g., to measure flow rate of the therapeutic gas and/or liquid) or immediately downstream from the heating/cooling device 30 (e.g., to measure the temperature of the therapeutic gas and/or liquid).

In another aspect, the second end portion 24 of the first conduit 22 can be operably connected or coupled to the aerosol-generating device 17. The aerosol-generating device 17 can generally include any device or apparatus configured to generate very fine liquid droplets (e.g., with an average particle diameter of less than 5 microns) for inhalation into the airway and/or lung(s) of a subject 12. In one example, the aerosol-generating device 17 can include a nebulizer. In another example, the aerosol-generating device 17 can include an atomizer. Unlike conventional aerosol-generating devices, however, aerosol-generating device 17 of the present disclosure can further include an aerosol-generating mechanism that includes a vortex chamber (not shown) and/or a vibratory mesh (not shown) to improve aerosolization of a therapeutic liquid or solution and thereby increase delivery efficiency of the aerosolized solution. Examples of therapeutic solutions or liquids that can be aerosolized are described below. The aerosol-generating device 17 of the present disclosure improves efficiency of the system by decreasing the required amount of a therapeutic liquid or solution needed to effectively treat a subject 12, as well as ensuring improved delivery of a therapeutic agent (e.g., a drug) relative to particle size. The aerosol-generating device 17 of the present disclosure is capable of atomizing, nebulizing, or mixing liquids and gases, as well as different gases. It will be appreciated that the aerosol-generating device 17 may additionally include a mechanism (not shown) for controlling the temperature of aerosol. In such instances, it may not be necessary to include the heating/cooling device 30 described above.

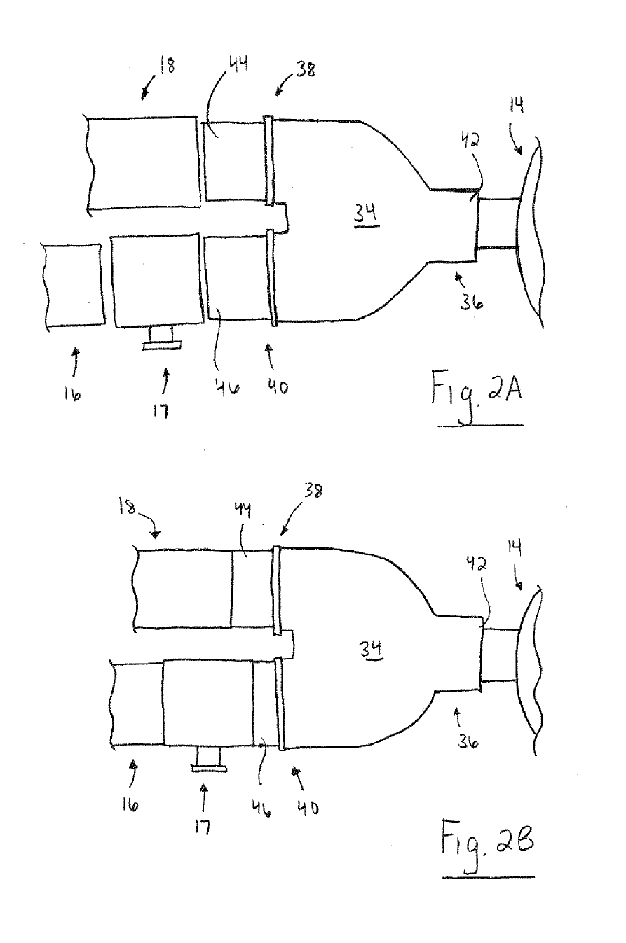

In another aspect, the aerosol-generating device 17 can be operably connected or coupled to the subject interface 14 via a Y-connector 34 (FIGS. 2A-B). The Y-connector 34 can be configured to facilitate inspiration of the aerosolized solution and expiration of exhaled gas. The Y-connector 34 can comprise a Y-shaped member including first, second, and third arms 36, 38 and 40. Each of the first, second, and third arms 36, 38, and 40 respectively includes first, second, and third ports 42, 44 and 46. The Y-connector 34 can have a rigid or semi-rigid configuration and be made, for example, from medical-grade plastic. As shown in FIGS. 2A-B, the first port 42 of the Y-connector 34 is configured to mate with the subject interface 14, the second port 44 is configured to mate with the expiratory limb 18, and the third port 46 is configured to mate with a portion of the aerosol-generating device 17. Although not shown in FIGS. 2A-B, it will be appreciated that a conduit (not shown) may extend between the aerosol-generating device 17 and the third port 46 of the Y-connector 34. In some instances, the Y-connector 34 can include an internal flow port or valve (not shown) configured to prevent the admixture of the inspiratory and expiratory flow streams, thereby reducing or preventing contamination (e.g., microbial contamination). For example, the flow port or valve can be integrated within a portion of the Y-connector 34. Moreover, the flow port or valve can be configured so as to not limit circulatory flow through the system 10 while also reducing or preventing contamination between the inspiratory and expiratory flow streams.

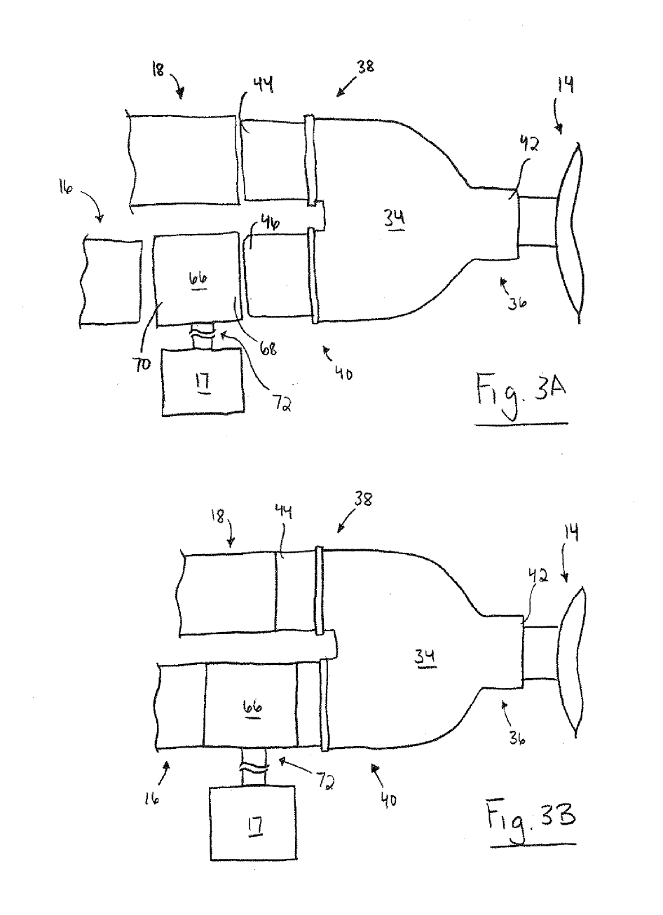

In another aspect, the inspiratory limb 16 can include an adaptor 66 (FIGS. 3A-B) configured to operably mate with an aerosol-generating device 17. As shown in FIGS. 3A-B, the adaptor 66 can have a generally cylindrical or tubular configuration with oppositely disposed first and second ends 68 and 70. The first and second ends 68 and 70 can be adapted to mate with the third port 46 and the second end portion 26 of the inspiratory limb 16, respectively. Although not shown, the adaptor 66 can include a lumen extending therethrough so that the inspiratory limb 16 and the Y-connector 34 are in fluid communication with one another when the system 10 is assembled. As also shown in FIGS. 3A-B, the adaptor 66 can include a port 72 configured to mate with a portion of the aerosol-generating device 17 so that the aerosol-generating device, when mated with the port, can flow an aerosolized fluid through the lumen of the adaptor and into the subject 12. The adaptor 66 can be rigid, semi-rigid, or flexible. All or only a portion of the adaptor 66 can be made from one or combination of suitable medical grade materials, such as plastic, stainless steel, glass, ceramics, and the like.

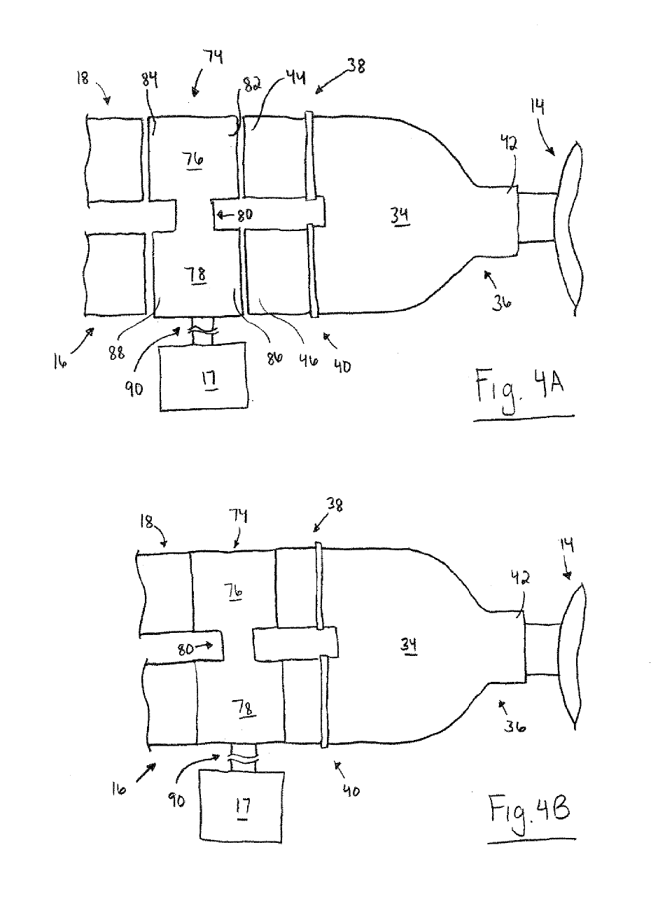

In another aspect, the inspiratory limb 16 can include an H-shaped adaptor 74 (FIGS. 4A-B) configured to join the inspiratory limb and the expiratory limb 18 in fluid communication with the Y-connector 34. As shown in FIGS. 4A-B, the H-shaped adaptor 74 includes oppositely disposed first and second arms 76 and 78 that are securely joined to one another by a bridge 80. Each of the first and second arms 76 and 78 can have a cylindrical or tubular configuration (e.g., similar or identical to the adaptor 66). The first arm 76 includes first and second ends 82 and 84 adapted to securely mate with the second port 44 of the Y-connector 34 and the first end portion 50 of the expiratory limb 18, respectively. The second arm 78 includes first and second ends 86 and 88 adapted to securely mate with the third port 46 of the Y-connector 34 and the second end portion 26 of the inspiratory limb 16, respectively. The second arm 78 additionally includes a port 90 configured to mate with a portion of the aerosol-generating device 17 so that the aerosol-generating device, when mated with the port, can flow an aerosolized solution through the lumen of the second arm into the subject 12. Each of the first and second arms 76 and 78 include a lumen (not shown). The lumen of each of the first and second arms 76 and 78 are not in communication with each other. The bridge 80 can have any configuration (e.g., length, width, diameter, etc.) and be solid, semi-solid or hollow. Additionally, the bridge 80 can be made of the same or different material(s) than the rest of the H-shaped adaptor 74. All or only a portion of the H-shaped adaptor 74 can be made from one or combination of suitable medical grade materials, such as plastic, stainless steel, glass, ceramics, and the like.

In another aspect, the subject interface 14 is operably connected or coupled to the first port 42 of the Y-connector 34 so that the first conduit 22 is in fluid communication therewith. The subject interface 14 can comprise any device or apparatus configured to facilitate delivery of the aerosolized solution into the airway and/or lung(s) of the subject 12. The subject interface 14 is also configured to facilitate expiration of exhaled gases into the expiratory limb 18 of the system 10. In some instances, the subject interface 14 can include a face mask (not shown). In other instances, the subject interface 14 can include an endotracheal tube (not shown). In further instances, the subject interface 14 can include a nasal cannula (not shown). Examples of face masks, endotracheal tubes, and nasal cannulas are known in the art.

In another aspect, the expiratory limb 18 comprises a second conduit 48 having oppositely disposed first and second end portions 50 and 52. In some instances, the second conduit 48 can comprise a flexible or flexibly resilient tube or line (e.g., plastic medical tubing) of a suitable length and size (e.g., diameter). The first end portion 50 of the second conduit 48 can be operably coupled or connected to the second port 44 of the Y-connector 34.

In some instances, the expiratory limb 18 can include at least one sensor 54 in electrical communication with the controller 20. The at least one sensor 54 can be configured to provide a feedback signal to the controller 20. In one example, the expiratory limb 18 can include a second sensor 54′ configured to measure the temperature of the exhaled gas from the subject 12. As described in more detail below, the sensed temperature of the exhaled gas can be relayed to the controller 20, which can then use the sensed temperature as a proxy for core body temperature to modulate other treatment parameters of the system 10. In other instances, the at least one sensor 54 of the expiratory limb 18 can measure the flow rate or the amount of the exhaled gas. Other parameters that may be detected by one or more sensors 54 can include gas concentration(s), expiratory limb pressure and vapor content, as well as other components of the exhaled gas, such as eicosanoids, vasoactive amines, cytokines, etc. It will be appreciated that the expiratory limb 18 can include any suitable number of sensors 54.

In another aspect, exhaled gas can pass through the second conduit 48 and out of the system 10 as indicated by arrow at second end portion of the second conduit. In some instances, a scavenger system (not shown) for capturing substances, including anesthesia gases and perfluorocarbons may be included as part of the expiratory limb 18. In other instances, the exhaled gases can be re-circulated back to ultimately feed the inspiratory limb 16 (e.g., after scrubbing and adding new gases), as indicated by the dashed line in FIG. 1. The expiratory limb 18 may additionally or optionally include a respiratory bag (not shown) that acts as a reservoir for respiratory gases, provides visual assessment of respiration, and serves as a mechanism to provide manual ventilation.

In another aspect, the system 10 can include a controller 20 in electrical communication with some or all of the system components. In some instances, the controller 20 can be in electrical communication with one or more components of the inspiratory limb 16, such as the source 28, the heating/cooling device 30, a sensor 32 (or sensors), and the aerosol-generating device 17. In other instances, the controller 20 can be in electrical communication with one or more components of the expiratory limb 18, such as the sensor(s) 54. The controller 20 can be configured to automatically regulate at least one treatment parameter based on feedback from one or more components of the system 10. In one example, the controller 20 can be configured to automatically regulate at least one treatment parameter based on a feedback signal (or signals) from one or more sensors 32 and/or 54.

In some instances, the controller 20 can include circuitry (hardware) configured to automatically regulate at least one treatment parameter. “Circuitry” can include electrical circuitry having at least one discrete electrical circuit, electrical circuitry having at least one integrated circuit, electrical circuitry having at least one application-specific integrated circuit, electrical circuitry forming a general purpose computing device configured by a computer program (e.g., a computer configured by a computer program that at least partially carries out processes described herein, or a microprocessor configured by a computer program that at least partially carries out processes described herein), electrical circuitry forming a memory device (e.g., forms of memory, such as random access, flash, read only, etc.), electrical circuitry forming a communications device (e.g., a modem, communications switch, optical-electrical equipment, etc.), and/or any non-electrical analog thereto, such as optical or other analogs. Those having skill in the art will recognize that the circuitry can be implemented in an analog fashion, a digital fashion, or some combination thereof.

In other instances, the controller 20 can include software configured to automatically regulate at least one treatment parameter. Software can generally include one or more computer programs and related data that provide instructions to the circuitry. The software can comprise one or more known types of software, such as system software (e.g., an operating system), programming software (e.g., defining the syntax and semantics of various programs), and application software (e.g., end-user applications). Other examples of software can include firmware, device drivers, programming tools, and middleware. In one example, software can include one or more algorithms configured to control heating and/or cooling of the aerosolized solution. In another example, software can include one or more algorithms configured to control the amount or rate of a therapeutic gas or liquid delivered to a subject 12.

It will be appreciated that the system 10 can include various other components to facilitate effective, closed-loop control and delivery of an aerosolized solution to a subject 12. In some instances, the system 10 can include a pump or compressor (not shown) to maintain flow rate and pressure in the system. In further instances, the system 10 can include a ventilator (not shown).

In one example, the inspiratory limb 16 can include a humidifier (not shown) for regulating moisture levels in the system 10. Various types of humidifiers are known in the art and can be integrated into the system 10. Examples of humidifiers that may be incorporated into the system 10 can include active humidifiers (e.g., low flow and high flow), passive humidifiers, wick humidifiers, vapor-phase humidifiers, and capillary force vaporizers. Active humidifiers, for example, use energy and water external to a patient's body for conditioning inspired gas, whereas passive humidifiers rely on temperature and humidity gradient between the patient's body and the external environment. Regardless of the type of humidifier, ventilation circuits that include a humidifier often suffer from build-up and persistence of harmful biofilms as a result of the warm, humid internal circuit environment. Because sterility of the ventilation circuit is paramount, biofilm formation often goes unchecked. In some instances, the system 10 can be advantageously configured for active humidification to prevent or mitigate biofilm formation. For example, the inspiratory limb 16 can include a humidifier placed downstream of a heating element 30 (e.g., distal to the gas or liquid source 28 and proximal to the aerosol-generating device 17). The heating element 30 can be configured to superheat the coolant or cryogen to a temperature (e.g., about 140° F.) sufficient to sterilize vapor or gas circulating in the system 10. As gas or vapor is circulated through the system 10, for example, the gas or vapor is superheated by the heating element 30 and then conditioned by the humidifier, thereby providing a mechanism for actively reducing or preventing biofilm formation in the system.

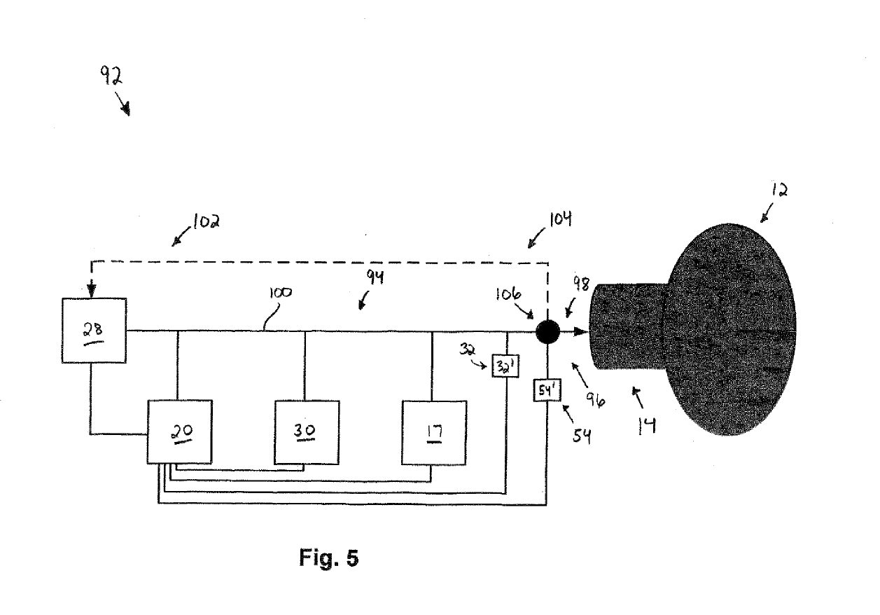

Another aspect of the present disclosure is illustrated in FIG. 5 and includes an alternative configuration of the system 10 (FIG. 1). In FIG. 5, a system 92 for treating a medical condition in a subject 12 using an aerosolized solution comprises certain components that are similar or identical to those in the system 10 (FIG. 1) described above. Thus, components of the system 92 (FIG. 5) that are same or similar to components of the system 10 (FIG. 1) will use the same reference numbers, whereas different components will use different reference numbers.

In one aspect, the system 92 (FIG. 5) can comprise a subject interface 14, an inspiratory limb 94 in fluid communication with the subject interface, an aerosol-generating device 17 operably coupled or connected to the inspiratory limb, an expiratory limb 96 in fluid communication with the subject interface, and a controller 20 in electrical communication with the inspiratory limb and the expiratory limb. The system 92 can further comprise a pressurized coolant or cryogen source 28, a heating and/or cooling device 30, at least one sensor 32 in electrical communication with the inspiratory limb 94, and at least one sensor 54 in electrical communication with the expiratory limb 96.

Unlike the system 10 in FIG. 1, which includes physically separated inspiratory and expiratory limbs 16 and 18, the inspiratory limb 94 and the expiratory limb 96 of the system 92 share a common portion 98 or pathway. As discussed in more detail below, the configuration of the system 92 can significantly increase the efficiency at which an aerosolized solution is delivered to a subject 12 by limiting or preventing the unwanted escape of the aerosolized solution from the system during exhalation. Consequently, the system 92 can significantly increase the amount of aerosolized solution inhaled by the subject while requiring less work of the system and/or its operator(s).

In one aspect, the inspiratory limb 94 can comprise a conduit 100 that is similarly or identically constructed as the first conduit 22 (FIG. 1). For example, the conduit 100 (FIG. 5) can comprise a flexible tube or line in fluid communication with the subject interface 14. In some instances, the conduit 100 can include a first end portion 102 and a second end portion 104. In other instances, the second end portion 104 can include a valve 106 that is operably secured thereto. The valve 106 can be located a suitable distance upstream (i.e., towards the first end portion 102). The section of the conduit 100 between the subject interface 14 and the valve 106 (inclusive) can define the common portion 98.

In some instances, the valve 106 can include at least one port (not shown) configured to convey an exhaled gas therethrough. Thus, the common portion 98, the valve 106, and the port can collectively define the expiratory limb 96. The valve 106 can be configured to permit passage of an aerosolized solution therethrough when a subject 12 inspires. During exhalation, the valve 106 can be configured to prevent passage of the aerosolized solution therethrough. Thus, the valve 106 can be configured to permit the passage of exhaled gas therethrough (e.g., through the port) when the subject 12 exhales without loss of the aerosolized solution. In one example, the valve 106 can comprise a one-way valve (e.g., a pop-off valve). In another example, the system 92 can include two one-way valves (not shown) such that a first valve opens during inspiration and shuts during expiration, while a second valve opens during expiration and shuts during inspiration.

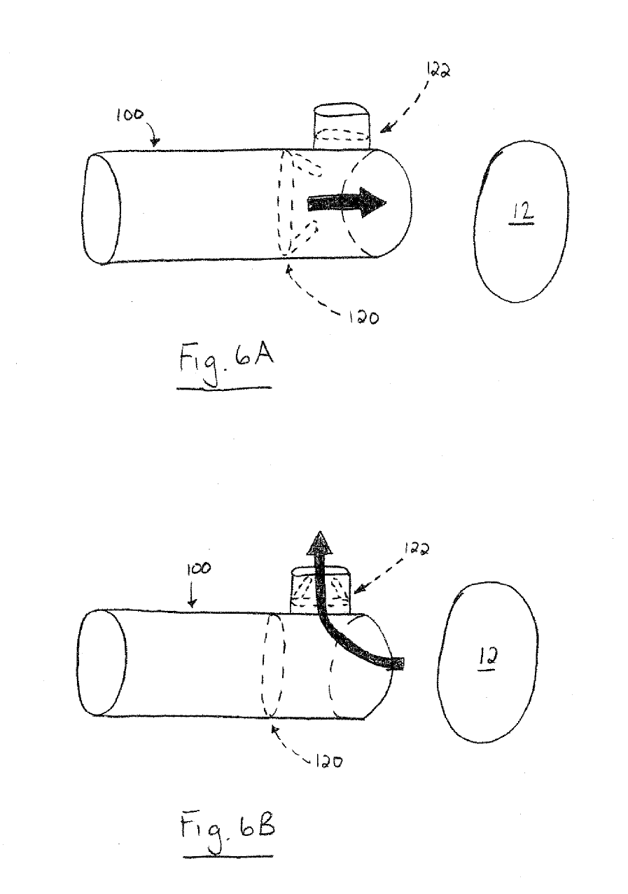

Another example of the valve 106 is shown in FIGS. 6A-B. The valve 106 can include first and second valves 120 and 122. Each of the first and second valves 120 and 122 can comprise a one-way valve. The first valve 120 can be operably disposed within the conduit 100. The second valve 122 can be operably integrated within the conduit 100 as well. During inspiration (FIG. 6A), the first valve 120 can open while the second valve 122 closes. This permits inhalation of an aerosolized solution (indicated by arrow) into the airway and/or lung(s) of the subject 12. During expiration (FIG. 6B), the first valve 120 can close while the second valve 122 opens. This permits release of exhaled gas from the system 92 (indicated by arrow) without concomitant release of the aerosolized solution, which is intended for inspiration.

In some instances, the valve 106 can be in electrical communication with the controller 20. In such instances, operation of the valve 106 can be automatically controlled based on one or more sensed parameters (e.g., temperature of the aerosolized solution, temperature of the exhaled gas, pressure, respiration rate, etc.). In other instances, operation of the valve 106 may be purely mechanical, meaning that the valve can function simply based on the breathing cycle of the subject 12.

In another aspect, exhaled gas can pass through the port of the valve 106 and out of the system 92. In some instances, a scavenger system (not shown) for capturing substances, including anesthesia gases and perfluorocarbons may be included as part of the expiratory limb 96. In other instances, the exhaled gas can be re-circulated back to ultimately feed the inspiratory limb 94 (e.g., after scrubbing and adding new gases), as indicated by the dashed line in FIG. 5. In such instances, the expiratory limb 96 may additionally or optionally include a respiratory bag (not shown) that acts as a reservoir for respiratory gases, provides visual assessment of respiration, and serves as a mechanism to provide manual ventilation.

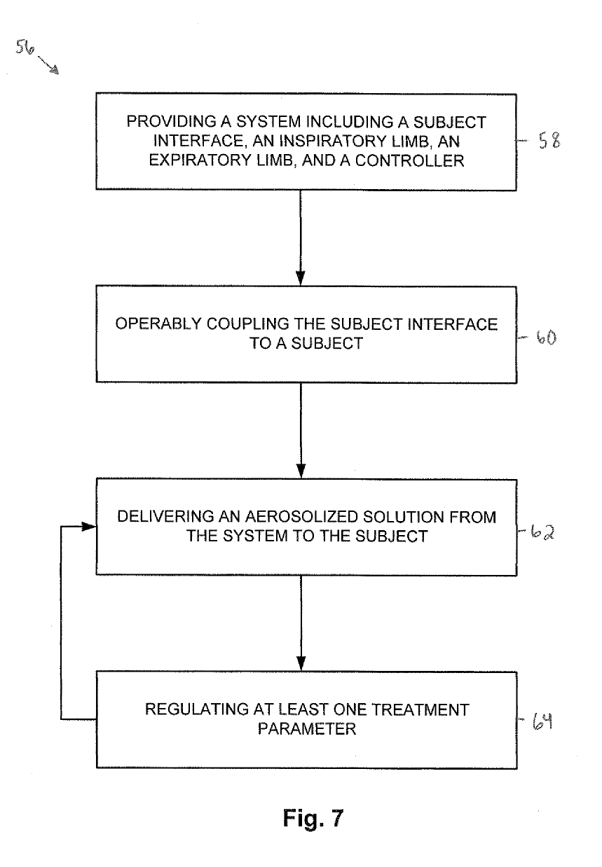

Another aspect of the present disclosure is illustrated in FIG. 7 and includes a method 56 for treating a medical condition in a subject 12. The method 56 includes the following steps: providing a system 10 (e.g., as shown in FIG. 1 and described above) (Step 58); operably coupling a subject interface 14 to a subject 12 (Step 60); delivering an aerosolized solution from the system to the subject (Step 62); and regulating at least one treatment parameter during delivery of the aerosolized solution to treat the subject (Step 64). The method 56 will be described below in terms of selectively modulating the temperature of a subject 12; however, as noted above, it will be appreciated that the method can be used to treat a variety of other medical conditions.

At Step 58, the system 10 can be optimally configured to induce therapeutic hypothermia in a subject 12 undergoing prolonged surgery. For example, the inspiratory limb 16 of the system 10 can be configured to include the following components: a pressurized source 28 of a coolant or cryogen; a heating/cooling device 30; a humidifier; an aerosol-generating device 17 (e.g., including a vortex chamber and/or vibratory mesh); and a first sensor 32′ configured to detect the temperature of the aerosolized solution being delivered to the subject. The expiratory limb 18 of the system 10 can be configured to include a second sensor 54′ configured to detect the temperature of exhaled gas from the subject 12. The controller 20 of the system 10 can be configured to include temperature regulatory algorithms for automatically controlling the core temperature of the subject 12 during treatment.

Once the system 10 is suitably configured, the system can be operably coupled to the subject 12 via the subject interface 14 (Step 58). Where the subject interface 14 comprises a face mask, for example, the face mask can be firmly secured about the head of the subject 12 so that the subject can easily inspire and expire.

At Step 62, operation of the system 10 can begin by flowing the coolant or cryogen through the inspiratory limb 16 in a desired amount (e.g., concentration) and at a desired rate. Delivery of the coolant or cryogen can be manually initiated (e.g., by actuating a valve on the coolant or cryogen source 28) or automatically initiated (e.g., by depressing a button). Alternatively, the desired rate of coolant or cryogen delivery into the inspiratory limb 16 can be controlled by the controller 20 (e.g., by pre-programming the controller). As the coolant or cryogen flows through the inspiratory limb 16, it can be cooled to a desired temperature by the heating/cooling device 30. The desired temperature of the cryogen or coolant can be preset by a user and/or selectively monitored and controlled by the controller 20. Next, the humidifier can add a desired amount of moisture into the system 10. The amount of moisture can be preset by a user and/or selectively monitored and controlled by the controller 20.

The coolant or cryogen next enters the aerosol-generating device 17, where it is mixed with a solution that includes at least one medication or drug (e.g., an atomized solution). The medication can include any substance or agent capable of preventing the body's reflex heat production mechanisms, which are activated when the body is cooled. This includes medications to suppress the thermoregulation center located in the brain and to suppress peripheral heat production in the skeletal muscles, liver, kidney, adipose tissue and other cellular structures. General anesthetics, narcotics, and anti-serotonin agents may be administered to suppress the central heat regulation center, while muscle relaxants, anti-thyroid agents and sympatholytic agents may be used to decrease peripheral heat production. It will be appreciated that the medication(s) or drug(s) aerosolized and delivered to the subject 12 can vary depending upon the medical condition being treated. For example, a chemotherapeutic agent can be aerosolized and delivered to a subject 12 suffering from cancer. Other examples of medications or drugs that may be mixed with an aerosolized solution can include antimicrobial agents (e.g., antiviral agents, antibiotics, antifungal agents, etc.), polynucleotides (e.g., gene therapy agents, such as siRNAs), polypeptides (e.g., biologics), and other small molecules suitably formulated for inhalation.

The resultant mixture of coolant or cryogen and aerosolized solution produces an aerosol that is inhaled by the subject 12. The degree to which a subject is cooled will be determined by clinical considerations on a case-by-case basis. The aerosolized solution may be administered at a temperature only slightly below body temperature, e.g., at 30° C. or, alternatively, at near-freezing temperatures. Generally speaking, hypothermia is achieved through loss of heat from the lungs. The large surface area of the pulmonary alveolus is utilized to exchange heat from the body to inspired gases. In the lungs, blood comes in close proximity to the inspired gases, being separated by the alveolar membrane only a few microns in thickness. The gossamer thinness and the large surface area of the lungs are ideally suited for heat exchange.

The subject 12 may be allowed to breathe spontaneously or, alternatively, the subject may be mechanically ventilated. While breathing, heat is transferred to the inspired gases, which is then carried away with the exhaled gases. This heat loss is further enhanced by lowering the temperature of the inspired gases. As per the laws of thermodynamics, the heat exchange between the blood in the lung alveoli and the inspired gases is directly proportional to the temperature difference between them. The temperature of the aerosolized solution can be monitored at the point of entrance into the respiratory system (e.g., by the first sensor 32′). The temperature of the aerosolized solution may be maintained automatically by the controller 20, which is in electrical communication with the first sensor 32′. For example, the temperature of the inspired aerosolized solution may be altered by automatically changing the settings on the heating/cooling device 30.

As the subject 12 exhales, the expired gas travels through the expiratory limb 18 of the system 10. The second sensor 54′ can then detect the temperature of the exhaled gas, which is indicative of the subject's core body temperature. Closed-loop control of thermal therapy, such as that provided by the system 10, requires feedback of a temperature signal, which represents the state of the subject 12 to which the therapy is applied. This signal, combined with the target temperature of the therapy, serves as an input for the controller 20, which can include a thermal control algorithm to regulate the energy added or removed from the subject 12. The detected temperature is then provided as feedback to the controller 20. Where the core body temperature needs to be lowered, the controller 20 may automatically change the settings on the heating/cooling device 30 so that the temperature of the aerosolized solution is further decreased. Alternatively, where the subject's core body temperature is too low, the controller 20 can automatically change the settings on the heating/cooling device 30 so that the temperature of the aerosolized solution is increased.

Either shortly before or once surgery is completed on the subject 12, the subject can be re-warmed by heating the inspired aerosolized solution. As stated by Fourier's Law, the heat flux is proportional to the magnitude of the temperature gradient and opposite in direction of flow. Hence, if the aerosolized solution is warmer than the body, heat will flow from the inspired aerosolized solution to the blood in the lungs. Warmed circulating blood will gradually warm the rest of the subject's body. Advantageously, the method 56 of the present disclosure provides precise, closed-loop control for effective delivery of an aerosolized solution to a subject 12 without the need for endovascular devices or cumbersome external components.

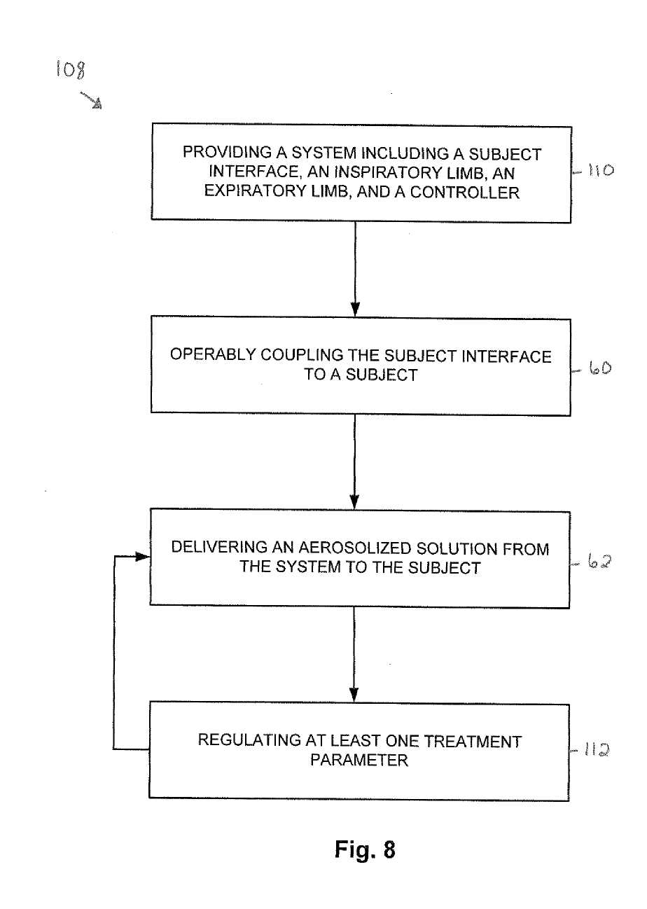

Another aspect of the present disclosure is illustrated in FIG. 8 and includes a method 108 for treating a medical condition in a subject 12. Steps of the method 108 that are identical or similar to those in FIG. 7 use the same reference numbers, whereas steps that are different from the method 56 use different reference numbers. The method 108 (FIG. 8) can include the following steps: providing a system 92 (e.g., as shown in FIG. 5 and described above) (Step 110); operably coupling a subject interface 14 to a subject 12 (Step 60); delivering an aerosolized solution from the system to the subject (Step 62); and regulating at least one treatment parameter during delivery of the aerosolized solution to treat the subject (Step 112). The method 108 will be described below in terms of delivering chemotherapy to a subject 12; however, as noted above, it will be appreciated that the method can be used to treat a variety of other medical conditions.

At Step 110, the system 92 can be optimally configured to deliver chemotherapy to the subject 12. For example, the inspiratory limb 94 of the system 92 can be configured to include the following components: a pressurized source 28 of a coolant or cryogen; a heating/cooling device 30; a humidifier; an aerosol-generating device 17 (e.g., including a vortex chamber and/or vibratory mesh); and a first sensor 32′ configured to detect the temperature of an aerosolized chemotherapeutic solution being delivered to the subject. The expiratory limb 96 of the system 92 can be configured to include a valve 106 having at least one port, and a second sensor 54′ configured to detect the temperature of exhaled gas from the subject 12. The controller 20 of the system 92 can be configured to include regulatory algorithms for automatically controlling the rate, temperature, and/or amount of the aerosolized chemotherapeutic solution delivered to the subject 12 during treatment.

Once the system 92 is suitably configured, the system can be operably coupled to the subject 12 via the subject interface 14 (Step 60). Where the subject interface 14 comprises a face mask, for example, the face mask can be firmly secured about the head of the subject 12 so that the subject can easily inspire and expire.

At Step 62, operation of the system 92 can begin by flowing the coolant or cryogen through the inspiratory limb 94 in a desired amount (e.g., concentration) and at a desired rate. Delivery of the coolant or cryogen can be manually initiated (e.g., by actuating a valve on the coolant or cryogen source 28) or automatically initiated (e.g., by depressing a button). Alternatively, the desired rate of coolant or cryogen delivery into the inspiratory limb 94 can be controlled by the controller 20 (e.g., by pre-programming the controller). As the coolant or cryogen flows through the inspiratory limb 94, it can be cooled to a desired temperature by the heating/cooling device 30. The desired temperature of the cryogen or coolant can be preset by a user and/or selectively monitored and controlled by the controller 20. Next, the humidifier can add a desired amount of moisture into the system 92. The amount of moisture can be preset by a user and/or selectively monitored and controlled by the controller 20.

The coolant or cryogen next enters the aerosol-generating device 17, where it is mixed with a solution containing at least one chemotherapeutic agent. The chemotherapeutic agent can include any agent that reduces, prevents, mitigates, limits, and/or delays the growth of metastases or neoplasms, or kills neoplastic cells directly by necrosis or apoptosis of neoplasms or any other mechanism, or that can be otherwise used, in a pharmaceutically-effective amount, to reduce, prevent, mitigate, limit, and/or delay the growth of metastases or neoplasms in a subject with neoplastic disease. Chemotherapeutic agents can include, for example: fluoropyrimidines; pyrimidine nucleosides; purine nucleosides; anti-folates, platinum complexes; anthracyclines/anthracenediones; epipodopodophyllotoxins; camptothecins; hormones; hormonal complexes; antihormonals; enzymes, proteins, and antibodies; vinca alkaloids; taxanes; antimirotubule agents; alkylating agents; antimetabolites; topoisomerase inhibitors; antivirals; and miscellaneous cytotoxic and cytostatic agents.

The resultant mixture of the coolant or cryogen and the aerosolized solution produces an aerosolized chemotherapeutic solution that can be inhaled by the subject 12. The subject 12 may be allowed to breathe spontaneously or, alternatively, the subject may be mechanically ventilated. During inhalation, the valve 106 can be actuated so that the aerosolized chemotherapeutic solution travels through the common portion 98 and into the subject's lung(s) via the subject interface 14. The temperature of the aerosolized chemotherapeutic solution can be monitored at the point of entrance into the respiratory system (e.g., by the first sensor 32′). The temperature of the aerosolized chemotherapeutic solution may be maintained automatically by the controller 20, which is in electrical communication with the first sensor 32′. For example, the temperature of the inspired aerosolized chemotherapeutic solution may be altered by automatically changing the settings on the heating/cooling device 30.

As the subject 12 exhales, the valve 106 can close when the exhaled gas moves through the common portion 98 towards the first end portion 102 of the conduit 100. With the valve 106 closed, the exhaled gas can travel through the port and out of the system 92. The second sensor 54′ can detect the temperature of the exhaled gas. The detected temperature can then be provided as feedback to the controller 20. The controller 20 can automatically change the settings on the heating/cooling device 30 so that the temperature of the aerosolized chemotherapeutic solution may be adjusted (e.g., increased or decreased).

The system 92 and method 108 can significantly increase delivery of an aerosolized solution to a subject 12. Conventional systems and methods for delivering an aerosolized solution automatically waste at least 50% of the aerosolized solution simply because flow of the aerosolized solution through the system is always on or constant. Consequently, a portion of the aerosolized solution that is intended for delivery to the lung(s) of a subject 12 never reaches its target because it is forced out of the system along with the exhaled gas during exhalation. Advantageously, the system 92 and method 108 can significantly increase (e.g., double) the effective dosage of the aersolized solution since the valve 106 prevents or limits constant flow of the aerosolized solution into the subject interface 14 while the subject exhales.

From the above description of the present disclosure, those skilled in the art will perceive improvements, changes and modifications. For example, it will be appreciated that one or more components of the present disclosure need not be in electrical communication with the controller 20. In such instances, it will be appreciated that components not in electrical communication with the controller 20 can function mechanically (e.g., as a result of pressure and/or temperature fluctuation); that is, without electrical actuation. Such improvements, changes, and modifications are within the skill of one in the art are intended to be covered by the appended claims.

The following is claimed:

1. A system for treating a medical condition in a subject using an aerosolized solution, said system comprising:
a subject interface;
an inspiratory limb in fluid communication with said subject interface, said inspiratory limb including an aerosol-generating device configured to provide the aerosolized solution;
an expiratory limb in fluid communication with said subject interface; and
a controller in electrical communication with said inspiratory and expiratory limbs, said controller configured to automatically regulate at least one treatment parameter based on feedback from one or more sensors operably integrated into said inspiratory limb and/or said expiratory limb, said at least one treatment parameter being selected from the group consisting of the amount, rate, and temperature of the aerosolized solution.

2. The system of claim 1, further comprising a Y-connector having a first port configured to mate with said subject interface, a second port configured to mate with an end of said expiratory limb, and a third port configured to mate with an end of said aerosol-generating device.

3. The system of claim 1, said inspiratory limb further including:
a coolant or cryogen source;
a humidifier; and
a first sensor for sensing the temperature of a coolant or cryogen being delivered to the subject.

4. The system of claim 1, said expiratory limb further including a second sensor for sensing the temperature of the gas being exhaled from the subject.

5. The system of claim 1, said expiratory limb comprising a valve in fluid communication with at least one port configured to convey an exhaled gas therethrough.

6. The system of claim 1, wherein said aerosol-generating device further includes an aerosol-generating mechanism to improve aerosolization of a therapeutic liquid or solution and increase delivery efficiency of the aerosolized solution into the subject.

7. The system of claim 1, wherein said aerosol-generating mechanism includes a vortex chamber, a vibratory mesh, or a combination thereof.

8. The system of claim 1, wherein said controller includes software configured to maintain the core temperature of the subject based on feedback from said one or more sensors.

9. A system for regulating the body temperature of a subject using an aerosolized solution, said system comprising:
a subject interface;
an inspiratory limb in fluid communication with said subject interface, said inspiratory limb including a aerosol-generating device configured to provide the aerosolized solution;
an expiratory limb in fluid communication with said subject interface;
a controller in electrical communication with said inspiratory and expiratory limbs, said controller configured to automatically regulate at least one treatment parameter based on feedback from one or more sensors operably integrated into said inspiratory limb and/or said expiratory limb, said at least one treatment parameter being selected from the group consisting of the amount, rate, and temperature of the aerosolized solution; and
a Y-connector having a first port configured to mate with said subject interface, a second port configured to mate with an end of said expiratory limb, and a third port configured to mate with an end of said aerosol-generating device;
wherein said aerosol-generating device further includes an aerosol-generating mechanism to improve aerosolization of a therapeutic liquid or solution and increase delivery efficiency of the aerosolized solution into the subject.

10. The system of claim 9, wherein said aerosol-generating mechanism includes a vortex chamber, a vibratory mesh, or a combination thereof.

11. The system of claim 9, said inspiratory limb further including:
a coolant or cryogen source;
a humidifier; and
a first sensor for sensing the temperature of a coolant or cryogen being delivered to the subject.

12. The system of claim 9, said expiratory limb further including a second sensor for sensing the temperature of the gas being exhaled from the subject.

13. The system of claim 9, said expiratory limb comprising a valve in fluid communication with at least one port configured to convey an exhaled gas therethrough.

14. The system of claim 9, wherein said controller includes software configured to maintain the core temperature of the subject based on feedback from said one or more sensors.

15. A method for treating a medical condition in a subject, said method comprising the steps of:
providing a system including a subject interface, an inspiratory limb in fluid communication with the subject interface, an expiratory limb in fluid communication with the subject interface, and a controller in electrical communication with the inspiratory and expiratory limbs;
operably coupling the subject interface to the subject; and
delivering an aerosolized solution from the system to the subject in an amount and for a time sufficient to treat the medical condition;
wherein the controller automatically regulates at least one treatment parameter based on feedback from one or more sensors operably integrated into the inspiratory limb and/or the expiratory limb, the at least one treatment parameter being selected from the group consisting of the amount, rate, and temperature of the aerosolized solution.

16. The method of claim 15, wherein delivery of the aerosolized solution induces and maintains hypothermia in the subject.

17. The method of claim 15, wherein the aerosolized solution is delivered to the subject following an ischemic event.

18. The method of claim 15, wherein the aerosolized solution is delivered to the subject to treat cancer.

19. The method of claim 15, wherein the aerosolized solution is a nebulized solution.

20. The method of claim 15, wherein the aerosolized solution is an atomized solution.
